(19) United States
(12) Patent Application Publication
(10) Pub. No.: US 2012/0172724 A1
(43) Pub. Date: Jul. 5, 2012

(54) AUTOMATIC IDENTIFICATION OF INTRACARDIAC DEVICES AND STRUCTURES IN AN INTRACARDIAC ECHO CATHETER IMAGE

(76) Inventors: Anthony D. Hill, Minneapolis, MN (US); D. Curtis Deno, Andover, MN (US); Hua Zhong, Pittsburgh, PA (US); Martin M. Grasse, Boston, MA (US)

(21) Appl. No.: 12/983,013

(22) Filed: Dec. 31, 2010

(51) Int. Cl. A61B 8/14

(52) U.S. Cl. 600/443

(57) ABSTRACT

An intracardiac imaging system configured to display electrode visualization elements within an intracardiac echocardiography image where the electrode visualization elements represent intracardiac electrodes in close proximity to the plane of the image. The system further allows cross sections of tissue structures embodied in intracardiac echocardiography images to be modeled within a visualization, navigation, or mapping system when automatically segmented to generate shell elements for modifying the modeled tissue structures.

### BACKGROUND OF THE INVENTION

a. Field of the Invention

The present invention relates to medical imaging and physiologic modeling, and particularly, the present invention relates to the identification and tracking of devices and structures within one imaging or modeling modality and the concurrent display of that information within a separate imaging or modeling modality.

b. Background Art

It is well known that the prevalence of different imaging modalities in medicine provide the clinician with valuable information regarding patient physiology. However, all imaging modalities suffer from some type of error that introduces uncertainty into the resulting images. Further limiting the usefulness of medical images is the difficulty in interpreting the content of an image when the image contains no identifiable landmarks to provide context. Without a landmark to locate an image within the body the content of the image can often be of limited use.

It is well known that intracardiac echo (“ICE”) catheters provide images of cardiac structures and, under some conditions, other intracardiac catheters. The metal electrodes present on intracardiac electrodes are very echogenic and produce bright signatures in echo images, particularly when the catheter shaft is not axially aligned with the echo plane or when the catheter's shaft lies in the plane of the echo beam and is oriented perpendicular to it. However, visual identification of another intracardiac catheter in the echo image often is not sufficient to locate the ICE catheter and does not allow the accurate combination of an ICE echo image and a geometric model created with electric or magnetic field modeling.

Another imaging modality commonly employed is three dimensional mapping using electrical or magnetic fields to create a geometric model. The geometric model is then constructed with reference to a static reference electrode. The reference electrode allows the mapping device to compensate for voluntary shifting by the patient, such as from localized discomfort, and to compensate for involuntary movement, such as breathing, thereby creating a more stable model. However, electrical navigational fields are not assured to be homogeneous or isotropic, so it is common for these geometric models to suffer from distortion. Further complicating the location of ICE catheters and the images they produce is the fact that the echo images often are not representative of the idealized echo plane, as it has been observed that the echo image commonly suffers from both rotational and translational deviations from the ideal.

For the foregoing reasons, there is a need for the combination of three dimensional cardiac models and ICE catheter images to provide more useful images to the clinician. It is desirable to be able to combine the imaging data so as to provide a more complete representation of patient physiology than is possible with a single imaging modality.

### BRIEF SUMMARY OF THE INVENTION

To this end, the present invention allows for the display of any electrophysiology procedure (“EP”) device tracked within a physiological visualization, navigation, or mapping system in an ICE echo image. Further, the present invention allows for the combination of structures or surfaces defined within a geometric model of a VNM system with the ICE echo image to refine the geometric model using the ICE echo image information.

Display of a tracked EP device within an ICE echo image allows the clinician to more easily navigate both the ICE catheter and other EP devices. A tracked EP device can have its position relative to the ICE echo plane calculated if the position of the ICE catheter within a geometric model maintained by the visualization, navigation, or mapping system is known. Any tracked EP device falling within or sufficiently close to the echo plane can then be displayed in the ICE image by a variety of visual identifiers.

The combination of ICE echo imaging information with structures from the geometric model allows the clinician to verify the position and structure of physical features and to identify errors in the geometric model. By projecting sectional representations from the geometric model into the ICE echo image discrepancies can be identified and corrected within the geometric model, thereby creating a more accurate model. Feature geometries from the geometric model projected into the ICE image are created by calculating the echo plane cross section of the feature and displaying the cross section boundaries within the ICE image. The portion of the ICE image falling within the cross sectional boundaries can then be segmented to separate tissue structures from voids, and the boundaries of the voids can then be displayed within the geometric model. By combining multiple segmented echo plane geometries a more complete model of the heart chamber can be acquired. The segmented chamber boundaries can also be used to create local deformations or modifications to the geometric model.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 depicts a block diagram generally illustrating the interrelationship of the various components of the system in an exemplary arrangement.

FIG. 2 generally depicts a two dimensional rendering of the geometric model illustrating a transformed intracardiac echo image in relation to electrodes having positions in close proximity to the echo image.

FIG. 3 generally depicts a two dimensional rendering of the geometric model illustrating an exemplary embodiment of an intracardiac echo image volume frame of the present disclosure.

FIG. 4 illustrates an exemplary embodiment of a user interface depicting an intracardiac echo image having electrode visualizations displayed therein in accordance with the present disclosure.

FIG. 5 illustrates an exemplary embodiment of a user interface depicting an intracardiac echo image having electrode visualizations and visual identifiers displayed therein in accordance with the present disclosure.

FIG. 6 illustrates an exemplary embodiment of a user interface depicting an intracardiac echo image having anatomic boundary references displayed therein in accordance with the present disclosure.

FIG. 7 depicts a diagrammatic illustration of an exemplary embodiment of the auto-segmentation algorithm in accordance with the present disclosure.

FIG. 8 illustrates an exemplary embodiment of a user interface depicting a shell element from an intracardiac echo image and a shell model displayed within the geometric model in accordance with the present disclosure.

### DETAILED DESCRIPTION OF THE INVENTION

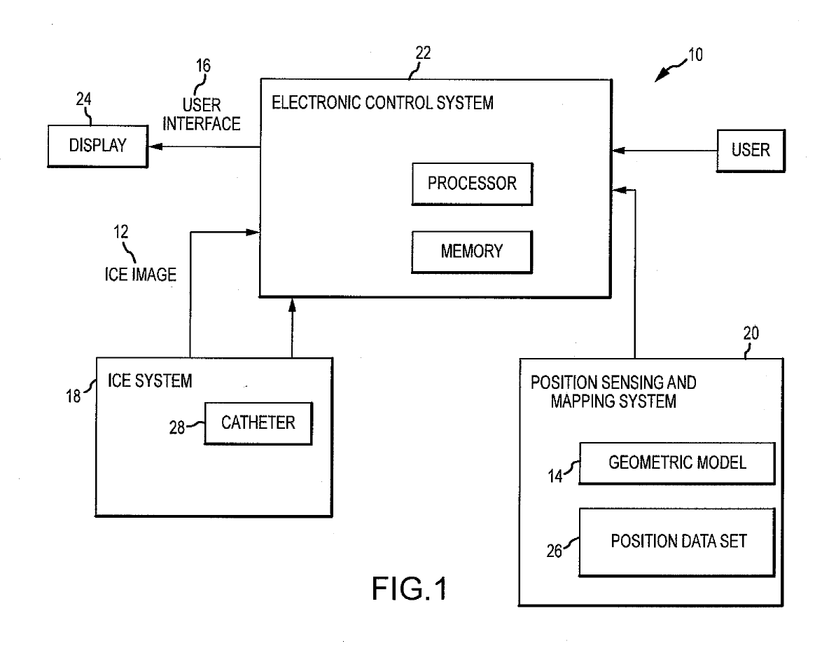

Referring now to the drawings wherein like reference numerals are used to identify identical components in the various views, FIG. 1 illustrates one exemplary embodiment of a system 10 configured to display within an intracardiac echocardiography image 12 (ICE image) devices present within a geometric model 14 of the heart and to auto-segment the ICE image 12 to generate one or more shell elements 36. The system 10 being further configured to generate a user interface 16 for displaying the ICE image 12 and the geometric model 14 as well as to receive user input directing the control and operation of the system 10.

The system 10 according to an embodiment of the present disclosure comprises an intracardiac echo imaging system 18 (ICE system), a visualization, navigation, or mapping system 20 (“VNM” system), an electronic control system (ECS) 22, and a display 24. The ECS 22 may be configured to receive an ICE image 12 produced by the ICE system 18 and the ECS 22 may further be configured to acquire a geometric model 14 of the heart and a position data set 26 from the VNM system 20. ECS 22 may be further configured to determine the position and orientation of the ICE image 24 within the geometric model 14 using the position data set 26 and to generate a user interface 16 containing the ICE image 12 having electrodes from the position data set 26 located in close proximity to the ICE image 12 depicted therein. The ECS 22 being further configured to execute an auto-segmentation routine to generate one or more shell elements 36 from the ICE image 12 for supplementing and displaying within the geometric model 14.

The ICE catheter 28 may contain a plurality of electrodes 30 or other sensors configured to be responsive to the VNM system 20 to allow the position and orientation of the ICE catheter 28, and thereby the ICE image 12, within the geometric model 14 to be determined. The ICE catheter 28 may contain three or more position sensors responsive to an electric or magnetic field generated by the VNM system 20, the sensors being positioned to define position and orientation of the ICE image plane when detected by the VNM system 20. An example of such an ICE catheter 28 is described in copending U.S. patent application Ser. No. 12/982,968 filed Dec. 31, 2010 entitled “INTRACARDIAC IMAGING SYSTEM UTILIZING A MULTIPURPOSE CATHETER,” which is hereby incorporated by reference in its entirety as though fully set forth herein.

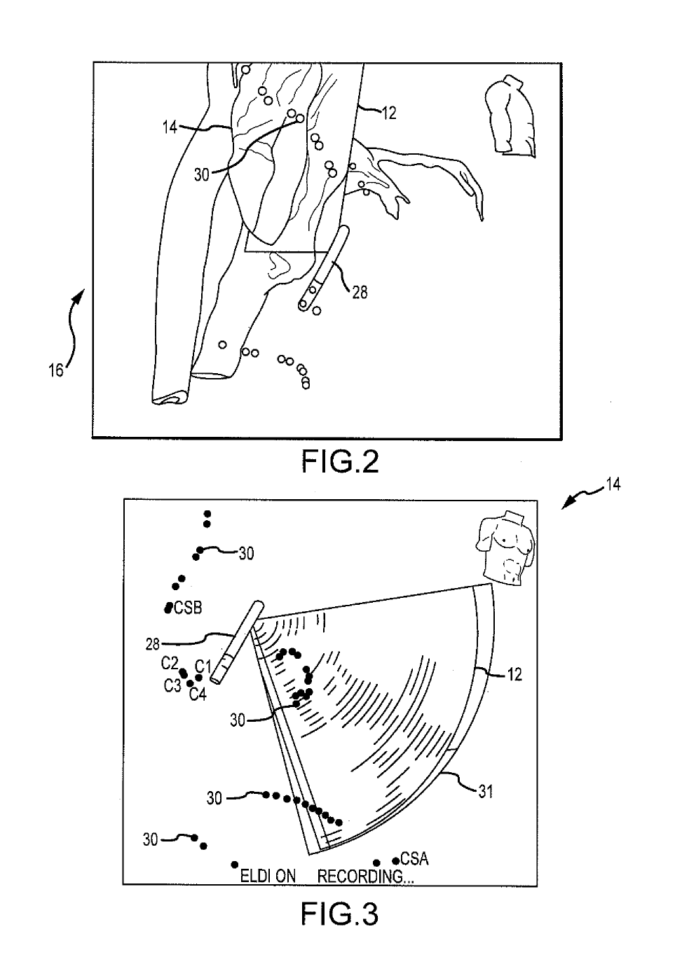

As depicted in FIG. 2, the ICE system 18 may be configured to produce an ICE image 12 that may be displayed within the user interface 16 on the display device 24. The ICE image 12 is generally fan shaped and depicts objects located within a plane of ultrasound energy emitted and received by the ICE catheter 28. ICE images 12 may be gray scale images with tissue structures, catheters and other dense objected being displayed in white, while dark portions of the image tend to represent cavity space filled with fluid. The more echogenic (e.g., the denser) a material is, the brighter its representation will be displayed in the image 12.

The ECS 22 is electrically coupled to (i.e., via wires or wirelessly) to the VNM system 20 that can be configured to generate and maintaining a geometric model 14 of a body structure. The VNM system 20 may further be configured to determine the positioning (i.e., determine a position and orientation (P&O)) of a sensor-equipped medical device and to track the location of the medical device as part of a position data set 26 having as a component a listing of the locations of detected medical device sensors, such as electrodes 30, within the geometric model 14. The VNM system 20 may further be configured to allow the user to identify features within the geometric model 14 and include the location as well as other information associated with the identified feature, such as an identifying label, within the position data set 26. By way of example, identified features may include ablation lesion markers or anatomical features such as cardiac valves. Elements within the position data set 26 (i.e., detected electrodes and/or identified features) are considered tracked elements. Such functionality may be provided as part of a larger visualization, navigation, or mapping system, for example, an ENSITE VELOCITY™ system miming a version of NavX™ software commercially available from St. Jude Medical, Inc., and as also seen generally by reference to U.S. Pat. No. 7,263,397 entitled “METHOD AND APPARATUS FOR CATHETER NAVIGATION AND LOCATION AND MAPPING IN THE HEART” to Hauck et al., owned by the common assignee of the present application, and hereby incorporated by reference in its entirety. The VNM system 20 may comprise conventional apparatus known generally in the art, for example, the ENSITE VELOCITY™ system described above or other known technologies for locating/navigating a catheter in space (and for visualization), including for example, the CARTO visualization and location system of Biosense Webster, Inc., (e.g., as exemplified by U.S. Pat. No. 6,690,963 entitled “SYSTEM FOR DETERMINING THE LOCATION AND ORIENTATION OF AN INVASIVE MEDICAL INSTRUMENT” hereby incorporated by reference in its entirety), the AURORA® system of Northern Digital Inc., a magnetic field based localization system such as the gMPS system based on technology from MediGuide Ltd. of Haifa, Israel and now owned by St. Jude Medical, Inc. (e.g., as exemplified by U.S. Pat. Nos. 7,386,339, 7,197,354 and 6,233,476, all of which are hereby incorporated by reference in their entireties) or a hybrid magnetic field-impedance based system, such as the CARTO 3 visualization and location system of Biosense Webster, Inc. (e.g., as exemplified by U.S. Pat. Nos. 7,536,218, and 7,848,789 both of which are hereby incorporated by reference in its entirety). Some of the localization, navigation and/or visualization systems may involve providing a sensor for producing signals indicative of catheter location and/or orientation information, and may include, for example one or more electrodes in the case of an impedance-based localization system such as the ENSITE™ VELOCITY™ system running NavX software, which electrodes may already exist in some instances, or alternatively, one or more coils (i.e., wire windings) configured to detect one or more characteristics of a low-strength magnetic field, for example, in the case of a magnetic-field based localization system such as the gMPS system using technology from MediGuide Ltd. described above.

Although the exemplary VNM systems 20 described above each maintain a geometric model 14 of the body cavity, acceptable alternative mapping devices for creating a geometric model of cardiac structures include magnetic resonance imaging (MR) and x-ray computed tomography (CT).

While each of the electric-impedance, magnetic field, and hybrid magnetic field-impedance based systems disclosed above can act as the VNM system 20 and remain within the scope and spirit of the present disclosure, the VNM system of the remaining discussion will be assumed to be an impedance based system for the purposes of clarity and illustration unless otherwise noted.

The ECS 22 may include a programmed electronic control unit (ECU) having a processor in communication with a memory or other computer readable media (memory) suitable for information storage. Relevant to the present disclosure, the ECS 22 is configured, among other things, to receive user input from one or more user input devices electrically connected to the system 10 and to issue commands (i.e., display commands) to the display 24 of the system 10 directing the depiction of the user interface 16. The ECS 22 may be configured to be in communication with the ICE imaging system 18 and the VNM system 20 to facilitate the acquisition of the ICE image 12, as well as the geometric model 14 and position data set 26. The communication between the ICE imaging system 18 and the VNM system 20 may be accomplished in an embodiment through a communications network (e.g., a local area network or the internet) or a data bus.

It should be understood that although the VNM system 20, the ICE system 18, and the ECS 22 are shown separately, integration of one or more computing functions may result in a system including an ECS 22 on which may be run both (i) various control and image formation functions of the ICE system 18 and (ii) the geometric modeling and position tracking functionality of the VNM system 20. For purposes of clarity and illustration only, the description below will be limited to an embodiment wherein the ECS 22 is configured to perform the location of the ICE image 12 within the geometric model 14 and apply one or more electrode visualizations or visual identifiers to the ICE image 12, as well as execute an auto-segmentation routine to generate one or more shell elements. It will be appreciated, however, that in other exemplary embodiments, the ECS 22 may be configured to generate the ICE image 12 from signals generated by the ICE catheter 28 and to generate the geometric model 14 from response signals generated by electrodes 30 within the body cavity being responsive to the electric or magnetic fields of the VNM system 20. This arrangement remains within the spirit and scope of the present disclosure.

As depicted in FIG. 3, the ECS 22 may generate and display a two dimensional rendering of the geometric model 14 within the user interface 16. The two dimensional rendering may display each tracked electrode 30 or identified feature within the position data set 26. The ECS 22 may locate and display the ICE image 12 within the geometric model 14 using data corresponding to the electrodes 30 defining the position and orientation of the plane of the ICE image 12 from the position data set 26, and if appropriate, display the ICE image 12 as part of the two dimensional rendering. Tracked electrodes 30 may have supplemental information in addition to location data associated with them in the position data set 26, such as electrode identifiers, associations with medical devices or other electrodes, and colors. FIG. 3 illustrates a two dimensional rendering of a geometric model 14 having an ICE image 12 located within the model 14 and several tracked electrodes 30 appearing as small colored spheres.

In an alternative embodiment, the two dimensional rendering of the geometric model 14 may contain an ICE image volume frame 31 depicting an approximation of the volume resolved within the ICE image 12. As illustrated in FIG. 3, the two dimensional ICE image 12 can be projected into the geometric model 14 as a perfect plane having no depth. ICE images, however, are not representative of a perfect plane, as ICE catheters generally can receive ultrasound energy from a narrow angle just out of plane and cannot differentiate slightly out-of-plane energy from in-plane energy. The result is a two dimensional ICE image representing a thin volume of space commonly depicted as a perfect plane. Because ICE catheters generally receive out-of-plane energy across an angle, the distance an object may be from the idealized plane and still appear within the ICE image increases in proportion to the distance from the ICE catheter. In the embodiment depicted in FIG. 3, the ICE image volume frame 31 depicts an approximation of the outer boundaries of the volume resolved within the ICE image 12. The ICE image volume frame 31 can aid users in understanding why or when an object will appear or not appear within the ICE image 12. In an alternative embodiment, the ECS 22 may be configured to receive input from a user directing the ICE image volume frame 31 to be hidden or removed from the two dimensional rendering of the geometric model 14.

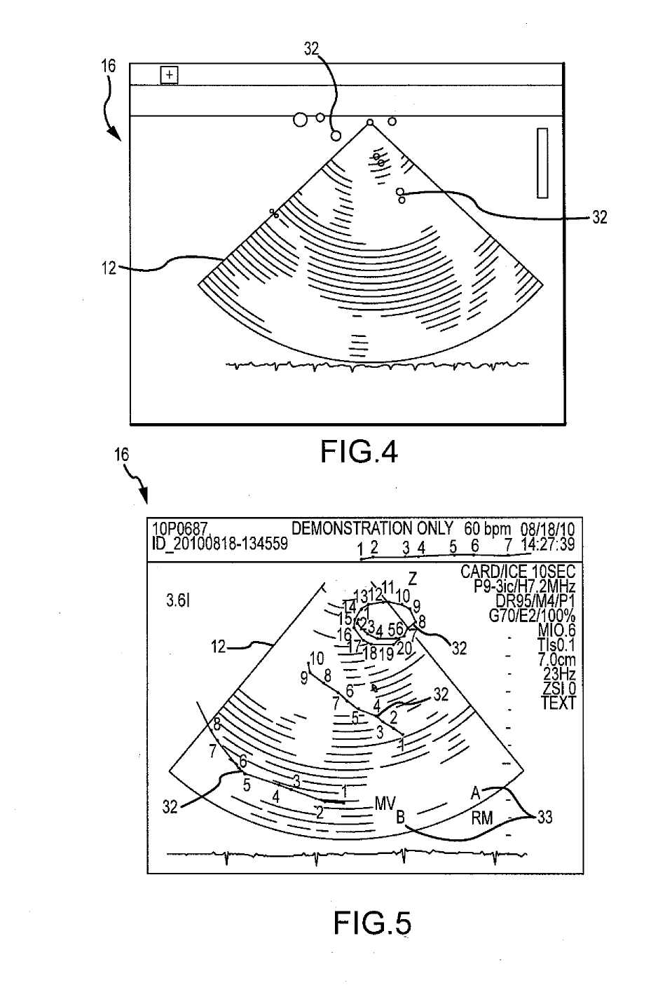

Display of a tracked electrode 30 or other tracked feature from the position data set 26 within the geometric model 14 within the ICE image 12 can be accomplished by locating the ICE catheter 28, and thereby the ICE image 12, within the geometric model 14, determining if any tracked electrode 30 or other tracked feature is intersected by the ICE image 12, and projecting an electrode visualization 32 for each intersected electrode 30 or visual identifier 33 for other intersected tracked features into the ICE image 12, embodiments of which is illustrated in FIGS. 4 and 5.

In an alternative embodiment consistent with the present disclosure, any electrode 30 in close proximity to the ICE image 12 may also be represented in the ICE image 12 by an electrode visualization 32. The proximity to the ICE image 12 threshold for display may be predetermined by the logic of the ECS 22, but may be adjusted by the user in an alternative embodiment. Visual identifiers 33 for other tracked features can be projected into the ICE image 12 regardless of the proximity of the tracked feature to the ICE image 12 in order to provide additional context for the ICE image 12.

In an alternative embodiment, the ECS 22 displays an electrode visualization 32 for any tracked electrode 30 intersected by a ray perpendicular to the ICE image 12, regardless of the position of the tracked electrode 30 relative to the ICE image 12 within the geometric model 14.

Projection of the electrode visualization 32 or visual identifier 33 into the ICE image 12 by the ECS 22 may be accomplished by transforming the position of the feature from the position data set 26 into a coordinate system of the ICE image 12, thereby adding the electrode visualization 32 or visual identifier 33 directly into the ICE image data. Alternatively, the electrode visualization 32 and/or visual identifier 33 can be superimposed on the ICE image 12 in the user interface 16. Transformation of the location of an electrode visualization 32 and/or visual identifier 33 from the coordinate space of the geometric model 14 to either the coordinate space of the ICE image 12 or the user interface 16 may be readily accomplished through matrix multiplication.

Although the above discussion focuses on projecting information from the geometric model 14 of the VNM system 20 into the ICE image 12. Once the ICE image 12 is located within the geometric model 14 information or features identified within the ICE image 12 may be projected into the geometric model 14, an example of which are shell elements that are described in detail below. Accordingly, such a projection from the ICE image 12 to the geometric model 14 remains within the scope of the present disclosure.

The electrode visualization 32 may take multiple forms, including a circle, as depicted by way of example in FIG. 4. In an embodiment, the color of the electrode visualization 32 is substantially the same as the color of the display of the corresponding tracked electrode 30 within the geometric model 14. The electrode visualization 32 of tracked electrodes 30 having supplemental information associated therewith may take a form depicting aspects of the supplemental information. In an exemplary embodiment illustrated in FIG. 5, the electrode visualization 32 can include an electrode identifier and lines showing associations with other tracked electrodes 30. For example, the electrodes 30 within a single catheter may have numeric electrode identifiers that correspond to their numbering in an EP recording system or in the VNM system 20. Electrodes 30 having an association within the supplemental information, such as, by way of example, being positioned adjacent to another electrode 30 within a single medical device, may be joined by colored line segments or other visual markers. Tracked electrodes 30 identified in this manner aid in user recognition and allow physicians to better assess the myocardial origin of electrogram signals that are associated with specific electrodes 30. An electrode visualization 32 in an alternate embodiment may take the form of an icon depicting a catheter or other medical device and further indicating the orientation of the device when known.

In an alternative embodiment, the ECS 22 may be configured to generate and display a color coded legend within the user interface 16 containing medical device names corresponding to the names from an EP recording system or VNM system 20. The electrode visualization 32 may also indicate whether an electrode 30 is directly intersected or located just outside the ICE image 12 by making slight variations in the size, color, or opacity of the electrode visualization 32. In an embodiment displaying supplemental information, including colored line segments to aid in the identification of catheters or other medical devices, variations in the opacity of the individual electrode visualizations 32 and associated line segments allow a user to visualize the orientation of the catheter relative to the echo image and anticipate what portion of the catheter displayed within the image 12 will be affected by contemplated changes in the position or orientation of the ICE catheter 28, the medical device, or both.

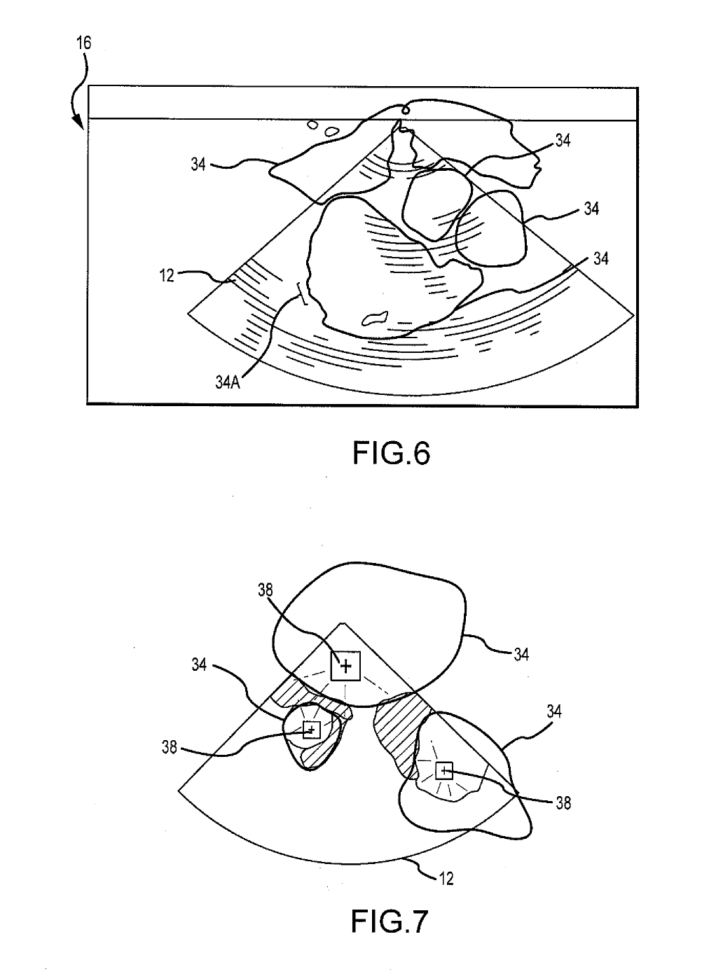

In addition to the tracked electrodes 30 discussed above, an anatomical boundary reference 34 produced by the intersection of the ICE image 12 with a boundary of the geometric model 14 may be projected into the ICE image 12, an exemplary embodiment of which is illustrated in FIG. 6. The anatomical boundary reference 34 is determined by locating the ICE image 12 within the geometric model 14, and calculating a cross section of any tissue structures intersected by the ICE image 12, where the perimeter points of the tissue cross section comprise the anatomical boundary reference 34. In an alternative embodiment, the geometric model 14 may be pre-segmented to delineate specific cardiac structures, such as heart chambers or vascular lumens, and these pre-segmented boundaries may be used as an anatomical boundary reference 34 when intersected by the ICE image 12. As with the electrode visualization 32, the anatomical boundary reference 34 may be transformed by the ECS 22 into either the ICE image 12 or superimposed upon the ICE image 12 displayed in the user interface 16 through matrix multiplication transformation between the coordinate systems.

In an alternative embodiment, anatomical boundary references 34 created from color coded pre-segmented cardiac structures and chambers can be projected into the ICE image 12 with the same color coding to aid in identification of an anatomical boundary reference 34. In yet another embodiment, the ECS 22 may be configured to generate and display within the ICE image 12 a color coded legend containing the colors and any associated labels from the pre-segmented chambers defining one or more anatomical boundary references 34. Projecting anatomical boundary references 34 further aids in navigation of the ICE catheter 28 and under certain circumstances allows for the modification of the geometric model 14, described below.

In yet another embodiment, the anatomical boundary references 34 may be displayed within the ICE image 12 in a manner depicting cardiac activity mapping. In such an alternative embodiment, the ECS 22 may acquire a cardiac activity map (i.e., activation timing or electrogram amplitude) for a pre-segmented portion of the geometric model 14 from, by way of example, the VNM system 20 or an external computer readable media in communication with the ECS 22. Cardiac activity maps can show varying levels activity using a spectrum or monochromatic variable color map where the activity is indicated by the color selected from either a multicolor or monochromatic scale. For example, a monochromatic scale uses variation in shade of a single color to indicate relative activity, such as white for the highest activity and black for no activity with a progressive shade change between the two bounds indicating gradations in activity. A spectrum map utilizes the dark to light bounds but varies the colors for the gradients in between the end bounds. For the purposes of this discussion the spectrum and monochromatic maps should be treated as interchangeable.

The portions of the cardiac activity map associated with the section of the pre-segmented geometry serving as the basis for the anatomical boundary reference 34 may be displayed as part of the anatomical boundary reference 34 within the ICE image 12. For example, the anatomical boundary references 34 shown in FIG. 6 would be depicted as a series of colored sub-elements 34a, where the color of each sub-element 34a is projected from the cardiac activity map, thereby displaying cardiac activity at the portion of the cardiac surface intersected by the ICE image 12. Display of the cardiac activity map information as part of the anatomical boundary reference 34 can help the user to locate and treat abnormal cardiac tissue.

When anatomical boundary references 34 are displayed in the ICE image 12 the portions of the ICE image 12 contained within the anatomical boundary references 34 may be used to generate one or more shell elements 36 generated by an auto-segmentation routine executed by the ECS 22. A diagrammatic illustration of an exemplary embodiment of the auto-segmentation algorithm is shown in FIG. 7. In an exemplary embodiment, the auto-segmentation algorithm selects a dark pixel from each portion of the ICE image 12 contained within an anatomical boundary reference 34. From the initial pixel, the auto-segmentation routine creates one or more void groups 38 by grouping any neighboring dark pixels with the initial pixel, and the routine continues adding pixels to the void group 38 until there are no more dark pixels adjacent to the void group 38. If additional dark pixels remain in the portion of the ICE image 12 contained within the anatomical boundary reference 34, then the algorithm selects an ungrouped dark pixel and repeats the grouping process, thereby creating another void group 38. The grouping process does not extend beyond the anatomical boundary reference 34, thereby limiting the segmentation process to known anatomical geometries. The segmentation is complete when all dark pixels have been assigned a void group 38, at which point all void groups 38 should be bounded by a light pixel edge or an anatomical boundary reference 34.

Delineation between dark and light pixels for the purpose of auto-segmentation can be accomplished in a variety of ways. In the one embodiment, the auto-selection algorithm sets the threshold as a percentage of the difference between the darkest pixel and lightest pixel in the ICE image 12. In another embodiment, the user interface 16 may be configured to receive user input directing the adjustment of the threshold value should the preset threshold of the auto-segmentation routine produce unsatisfactory results.

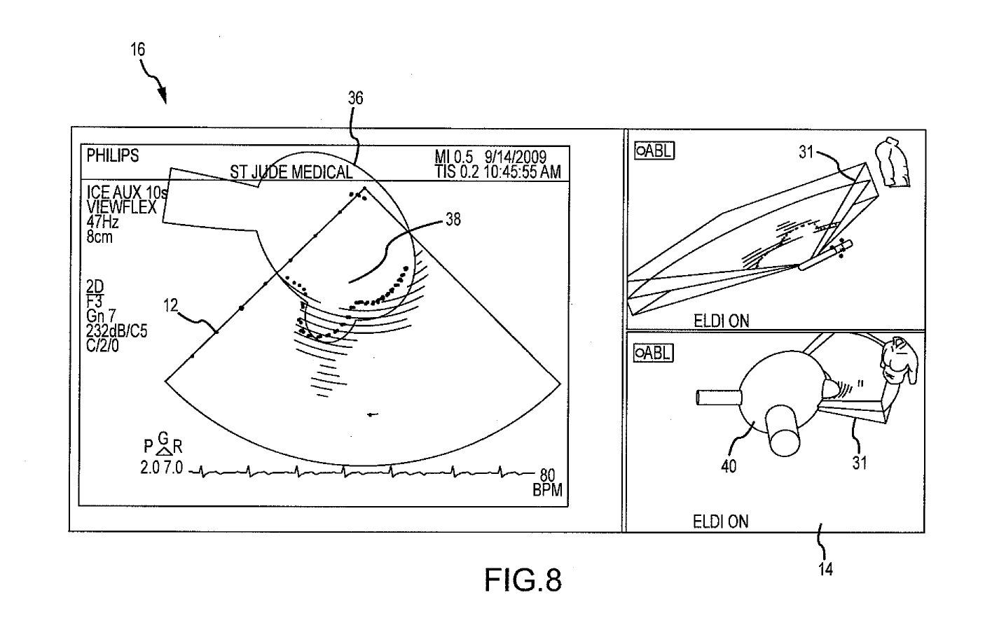

In an embodiment of the invention illustrated in FIG. 8, the perimeter of each void group 38 may form a shell element 36 that may be transformed to the geometric model 14 through matrix multiplication by the ECS 22. Displaying the shell element 36 in the geometric model 14 depicts the cardiac chamber boundary detected by the ICE system 18 through the ICE image 12. Optionally, the shell element 36 may be labeled with the anatomical boundary reference 34 bounding or initiating the auto-segmentation that produced the shell element 36. In an alternative embodiment, transformation of the shell element 36 into the geometric model 14 may allow the ECS 22 to create a deformation or modification of one or more anatomical features within the geometric model 14. Creation of deformations using shell elements 36 representing cross sections of the ICE image 12 allows the geometric model 14 to incorporate additional detail in areas of interest. A three dimensional shell model 40 may be generated by combining several shell elements 36 from several ICE images 12 produced from varying angles within an area of interest. The shell model 40 may be displayed within the geometric model 14 or incorporated into the model 14 as a modification to produce a more detailed geometry.

Although several embodiments of this invention have been described above with a certain degree of particularity, those skilled in the art could make numerous alterations to the disclosed embodiments without departing from the spirit or scope of this invention. For example, other algorithms can be used in place of the preferred auto-segmentation algorithm of the described embodiment to create pixel groupings. It is intended that all matter contained in the above description or shown in the accompanying drawings shall be interpreted as illustrative only and not limiting. Changes in detail or structure may be made without departing from the spirit of the invention as defined in the appended claims.

1. A visualization and modeling system comprising:
an ultrasound echo imaging system having an intracardiac echo catheter configured to produce a intracardiac echocardiography image (ICE image);
a visualization, navigation, or mapping system configured to generate a geometric model of a body cavity and generate a first position within the geometric model of the intracardiac echo catheter and a second position within the geometric model of a sensor of a medical device within the body cavity; and
an electronic control system (ECS) being configured to receive the ICE image, the geometric model, the first position, and the second position, the ECS being further configured to orient the ICE image within the geometric model at the first position, and generate a composite image when the oriented ICE image intersects the second position.

2. The system of claim 1, the composite image comprising a sensor visualization disposed on the ICE image.

3. The system of claim 2, further comprising a display device configured to be in communication with the ECS, the ECS being further configured to generate a user interface containing the composite image.

4. The system of claim 3, the user interface further containing a two dimensional rendering of the geometric model.

5. The system of claim 4, the two dimensional rendering containing an ICE image volume frame.

6. The system of claim 2, the ECS being further configured to generate a composite image when the ICE image located in the geometric model is within a threshold distance of the second position.

7. The system of claim 6, wherein the threshold distance is predetermined by the ECS.

8. The system of claim 6, wherein the threshold distance may be adjusted by the user.

9. The system of claim 1, the visualization, navigation, or mapping system being configured to generate a position data set comprising a plurality of sensor positions and sensor associations, each of the plurality of sensor positions corresponding to a location of a sensor of a medical device within the geometric model;
the composite image containing a sensor visualization depicting the position and associations of each member of the position data set having a position within a threshold distance of the first position.

10. The system of claim 9, the sensor visualization depicting the sensor associations as a line connecting at least two sensor locations.

11. A visualization and modeling system comprising:
an ultrasound echo imaging system having an intracardiac echo catheter (ICE catheter), the ultrasound echo imaging system being configured to generate a two-dimensional echocardiography image (ICE image);
a visualization, navigation, or mapping system configured to generate a geometric model and to determine the position and orientation of the ICE catheter within the geometric model; and
an electronic control system (ECS) configured to locate the ICE image within the geometric model and to execute an auto-segmentation routine generating a shell element, the electronic control unit further being configured to transform the shell element into the geometric model.

12. The system of claim 11, the geometric model being further configured to have a plurality of defined anatomic boundaries, the auto-segmentation routine being configured to generate a shell element partially bounded by at least one of the plurality of defined anatomic boundaries.

13. The system of claim 11, the auto-segmentation routine comprising the steps of:
selecting a dark pixel from a portion of the ICE image contained within the defined anatomic boundary;
creating a void group by adding to the void group all dark pixels adjacent to one of the selected dark pixel or another dark pixel within the void group.
creating a shell element.

14. The system of claim 13, the step of creating a shell element comprising selecting the perimeter pixels of the void group.

15. The system of claim 11, wherein the ECS is further configured to modify the geometric model to incorporate the shell element.

16. A method of enhancing a geometric model of a body cavity comprising the steps of:
acquiring a geometric model of a heart;
acquiring an intracardiac echocardiogram image (ICE image);
locating and orienting the ICE image in the geometric model;
segmenting the ICE image to produce a shell element; and
transforming the shell element into the geometric model.

17. The method of claim 15, further comprising the step of modifying the geometric model to incorporate the shell element.

18. The method of claim 15, further comprising the step of displaying a defined anatomic boundary within the ICE image from the geometric model.

19. The method of claim 18, the geometric model containing a pre-segmented chamber, wherein the defined anatomic boundary comprises the edge of the pre-segmented chamber.

20. The method of claim 15, wherein segmenting the ICE image comprises the steps of:
selecting a dark pixel from a portion of the ICE image contained within the defined anatomic boundary;
creating a void group by adding all dark pixels adjacent to one of the selected dark pixel or another dark pixel within the void group.
generating a shell element from the perimeter pixels of the void group.
